(19) United States
(12) Patent Application Publication
(10) Pub. No.: US 2004/0220433 A1
(43) Pub. Date: Nov. 4, 2004

(54) Process for the preparation of propylene glycol

(76) Inventors: Evert Van Der Heide, Amsterdam (NL); Jean-Paul Lange, Amsterdam (NL); Arjen Miedema, Amsterdam (NL)

Correspondence Address: Jennifer D. Adamson, Shell Oil Company, Legal - Intellectual Property, P.O. Box 2463, Houston, TX, 77252-2463, US

(21) Appl. No.: 10/802,630

(22) Filed: Mar. 17, 2004

(30) Foreign Application Priority Data
Mar. 28, 2003 (EP) 03251986.0

(51) Int. Cl. C07C 29/10

(52) U.S. Cl. 568/867

(57) ABSTRACT

The invention relates to a process for the preparation of propylene glycol from propylene oxide, which process involves:

(a) contacting propylene oxide with carbon dioxide in the presence of catalyst to obtain a first reaction mixture containing propylene carbonate, and

(b) contacting at least part of the first reaction mixture with water in the presence of catalyst to obtain a second reaction mixture containing propylene glycol and carbon dioxide,

in which process a substantial amount of propylene oxide is present in step (b).

### FIELD OF THE INVENTION

The present invention relates to a process for the preparation of propylene glycol from propylene oxide.

### BACKGROUND OF THE INVENTION

A cyclic alkylene carbonate may be obtained by contacting alkylene oxide with carbon dioxide in the presence of a suitable catalyst. Such process has been described for example in EP-A-119840. The cyclic alkylene carbonate can be converted further by hydrolysis of the cyclic alkylene carbonate to produce a diol as is described in U.S. Pat. No. 5,847,189.

A route for the preparation of monoethylene glycol comprises reacting ethylene oxide with carbon dioxide in water as described in U.S. Pat. No. 6,080,897 and U.S. Pat. No. 6,187,972. As mentioned in EP-A-1125915, the presence of ethylene oxide during hydrolysis of ethylene carbonate has the disadvantage that it can lead to the formation of by-products such as diethylene glycol.

### SUMMARY OF THE INVENTION

The present invention is directed to a process for the preparation of propylene glycol from propylene oxide, which process comprises (a) contacting propylene oxide with carbon dioxide in the presence of catalyst in the substantial absence of water to obtain a first reaction mixture containing propylene carbonate, and (b) contacting at least part of the first reaction mixture with water in the presence of catalyst to obtain a second reaction mixture containing propylene glycol and carbon dioxide, in which process a substantial amount of propylene oxide is present in step (b).

### DETAILED DESCRIPTION OF THE INVENTION

It was found that the presence of a substantial amount of propylene oxide during the hydrolysis of the propylene carbonate does not give substantial amounts of by-products. This is surprising as the reaction of propylene oxide with water in the presence of catalyst to obtain 1,2-propanediol is known to give substantial amounts of dipropylene glycol if no further compounds are present. It has now been found that propylene glycol can be manufactured with less by-product formation than formed in a process in which both water and carbon dioxide are present at the start while consuming less energy than a process in which carbonylation and hydrolysis are carried out strictly separately.

An advantage of the conversion of propylene oxide with water into 1,2-propanediol in the presence of propylene carbonate is that this reaction generates heat which can be used in the endothermic conversion of the propylene carbonate. Therefore, less cooling is needed during the conversion of propylene oxide while less heating can be carried out during the conversion of the propylene carbonate.

A further advantage of the presence of propylene oxide in the hydrolysis of the propylene carbonate is the fact that it is not required to remove all propylene oxide from the propylene carbonate before further conversion such as by full conversion of the propylene oxide.

In process step (a) of the present invention, the propylene oxide is contacted with carbon dioxide in the presence of catalyst. Several catalysts are known to be suitable for such process. Preferably, the catalyst is a homogeneous catalyst, more specifically a phosphorus containing homogeneous catalyst. Phosphorus containing compounds which are suitable catalysts are phosphine compounds and phosphonium compounds. The catalyst preferably is a homogeneous phosphonium catalyst, more specifically a phosphonium halide catalyst. It was found especially advantageous to employ a tetraalkylphosphonium halide catalyst, more specifically a tributyl-methyl phosphonium iodide.

The catalyst may either be added as such or can be formed in-situ.

The amount of water present in step (a) is at most limited. Generally, less than 1 mole of water per mole of propylene oxide is present, more specifically less than 0.5, more specifically less than 0.2, more specifically less than 0.1, most specifically les than 0.01.

The carbon dioxide may either be pure carbon dioxide or carbon dioxide containing further compounds. Carbon dioxide which is especially suitable for use in the present invention, is carbon dioxide which has been separated off in subsequent steps of the present process. Carbon dioxide may either be separated off directly after the propylene oxide has reacted with carbon dioxide or at a later stage.

Carbon dioxide is produced in the reaction of the propylene carbonate with water. Therefore, it is especially attractive to separate carbon dioxide and recycle the carbon dioxide thus obtained to step (a) either as such or after having been purified. The extent to which the carbon dioxide is purified depends on the nature and the amounts of contaminants present in the carbon dioxide. These again depend on the exact reaction conditions and purification steps of the process.

The propylene oxide may be reacted with carbon dioxide at suitable operating conditions. Such process conditions will generally comprise a temperature of from 50° C. to 200° C., more specifically of from 100° C. to 150° C. The pressure generally will be at least 5×10⁵ N/m², more specifically the pressure will generally be of from 5 to 100×10⁵ N/m², preferably of from 8 to 50×10⁵ N/m², more preferably of from 10 to 30×10⁵ N/m².

The catalyst may be added to the reactor in any form known to be suitable to someone skilled in the art. Generally, the catalyst will be added as such or as a solution of the catalyst preferably in a solvent such as a propylene carbonate or propylene glycol. The catalyst may be added either to the propylene oxide or to the carbon dioxide or to the mixture of both. Preferably, the catalyst solution is added to the reactor containing the mixture of propylene oxide and carbon dioxide.

The reaction mixture obtained in step (a) may be used without further purification in the manufacture of propylene glycol. However, some purification of the reaction mixture may be carried out. A purification which may be advantageous is the removal of at least part of the carbon dioxide from the reaction mixture obtained in step (a) before subjecting the remainder of the reaction mixture to step (b). Such purification may substantially reduce the volume of the reaction mixture to be subjected to step (b).

The first reaction mixture to be subjected to step (b) and referred to in the present invention, may either be the first reaction mixture obtained in step (a) which has not been treated further, or the first reaction mixture of step (a) which has been treated further in step (b), or a mixture of both the product of step (a) and the product of step (b). As mentioned hereinbefore, it is preferred that at least, part of the first reaction mixture is subjected to step (b) before sending it to step (b).

Process step (a) is preferably carried out with a homogeneous catalyst while step (b) is carried out with a heterogeneous catalyst. It has been found to be especially advantageous if the homogeneous catalyst for process step (a) is present in step (b). Without wishing to be bound to any theory, it is thought that the presence of the catalyst for process step (a) reduces the amount of by-products formed in the conversion of propylene oxide to propylene glycol in step (b). Removal of a limited amount of the homogeneous catalyst can occur during distillation or further processing of reaction mixture. However, such processes generally will leave sufficient homogeneous catalyst in the reaction mixture to serve its purpose in step (b) of the present process. A further improvement was observed if the homogeneous catalyst was present in combination with a substantial amount of carbon dioxide. Therefore, it is preferred in the present process to send the first reaction mixture directly from step (a) to step (b) while removing at most part of the carbon dioxide still present. It was found that such set-up gave less by-products such as dipropylene glycol.

The homogeneous catalyst which is preferably present in the crude reaction product of step (b), may be separated off from the second reaction mixture and recycled for use in step (a). The catalyst may be recycled in combination with further compounds either added to or formed in the process according to the present invention. Usually, the catalyst will be recycled while being dissolved in unconverted propylene carbonate.

A substantial amount of propylene oxide may be present in step (b). Preferably, the amount of propylene oxide and propylene carbonate which is present in step (b) is such that the molar ratio of propylene oxide to propylene carbonate is from 0.01 mole of propylene oxide per mole of propylene carbonate to 1 mole of propylene oxide per mole of propylene carbonate, i.e. from 0.01:1 to 1:1, more preferably from 0.02:1 to 0.6:1, more preferably from 0.03:1 to 0.4:1, more preferably from 0.04:1 to 0.3:1, more preferably from 0.05:1 to 0.2:1. Most preferably, the molar ratio of propylene oxide to propylene carbonate is from 0.08:1 to 0.15:1.

The first reaction mixture obtained in step (a) may contain the desired amount of propylene oxide due to the fact that part of the propylene oxide has not been converted in step (a) and/or propylene oxide can be added in step (b).

In a preferred embodiment, part of the propylene oxide which is present in step (a) is not converted in step (a) and is present in the feed of step (b). The exact amount of propylene oxide which is not converted, may vary widely as further propylene oxide may be added in process step (b). If no further propylene oxide is added in step (b), it is preferred that from 60% to 99% of the propylene oxide present in the feed of step (a) is converted in step (a). More specifically, from 60% to 95% of the propylene oxide present in the feed of step (a) is converted in step (a) in this embodiment, most specifically of from 70% to 90% wt. This preferred embodiment has the advantage over a conventional set-up in that the reactor for step (a) can be smaller than for a conventional process as the complete conversion of propylene oxide does not need to be ensured while the capacity can be reduced of both the cooling equipment for step (a) and the heating equipment for step (b).

The most preferred embodiment of the present invention comprises converting the propylene oxide present in the feed of step (a) substantially fully in step (a), and adding additional propylene oxide in step (b). The substantially full conversion in step (a) means that the majority of the propylene oxide is converted in step (a), more specifically at least 80% of the propylene oxide is converted. The addition of additional propylene oxide may be carried out before and/or during step (b). This set-up has the advantage that the propylene oxide may be added during step (b) such that an optimum temperature profile is attained over the reactor for step (b). In such case, it will usually be preferred to add the additional propylene oxide partly to the first reaction mixture before step (b) and partly during the conversion of the first reaction mixture in step (b). In a further preferred embodiment, the propylene oxide added to step (b) is added at different stages of conversion of step (b). Such addition allows optimum use of the heat generated by the hydrolysis of propylene oxide in step (b).

In step (b) of the present invention, the propylene carbonate is contacted with water. The heterogeneous catalysts for use in such process are known in the art. Examples of such catalysts comprise solid inorganic compounds such as alumina, silica-alumina, silica-magnesia, aluminosilicate, gallium silicate, zeolites, metal-exchanged zeolites, ammonium-exchanged zeolites, zinc on a support, lanthanum on a support, a mixture of aluminium and magnesium (hydr)oxide and ion-exchange resins.

Preferably, the heterogeneous catalyst employed in step (b) is chosen from the group consisting of a mixture of aluminium and magnesium (hydr)oxide, zinc on a support, lanthanum on a support and alumina. These catalysts will be described hereinafter in more detail.

The mixture of aluminium and magnesium (hydr)oxide preferably has a magnesium to aluminium molar ratio in the range of from 3 to 50, more preferably of from 4 to 20. In the preparation of the catalyst, generally a so-called mixed magnesium/aluminium hydroxide is formed. However, under working conditions mixed magnesium/aluminium oxides may be present. Our reference to a mixture of aluminium and magnesium (hydr)oxide covers both mixtures of aluminium and magnesium hydroxide and mixtures of aluminium and magnesium oxide and a combination of both mixtures. These mixtures were found to give the highest activity at a molar ratio of more than 3, preferably more than 4. A preferred range is from 4 to 20, more specifically from 5 to 15, most specifically from 5 to 10. Preferred catalysts are described in International application No. PCT/EP02/12640.

In another preferred embodiment of the present invention, the catalyst comprises a lanthanum compound on a support. A preferred catalyst comprises at least 7% wt of lanthanum supported on a support. The lanthanum compound preferably is La₂O₃ or a precursor thereof. Under reaction conditions this lanthanum compound may be temporarily and/or reversibly converted due to the reaction conditions into lanthanum hydroxide (La(OH)₃), lanthanum-oxyhydroxide (LaO(OH))and/or corresponding alcoholate species such as (La(OR)₃ or LaO(OR)).

As a support for the lanthanum containing catalyst any suitable support may be used. The support preferably is substantially inert under the reaction conditions and is provided with sufficient mechanical strength. Potential supports comprise clay minerals, inorganic supports such as Al₂O₃, SiO₂, MgO, TiO₂, ZrO₂, ZnO and mixtures thereof. Other examples are a kaolinite, a hallosyte, a chrysotile, a montmorillonite, a beidellite, a hectorite, a sauconite, a muscovite, a phlogopite, a biotite, a hydrotalcite and talc. Particularly preferred are the inorganic supports selected from the group consisting of Al₂O₃, SiO₂, MgO, TiO₂, ZrO₂, ZnO and mixtures thereof.

The lanthanum containing catalyst preferably comprises at least 7% wt of lanthanum, more specifically in the range of from 7% to 40% wt of lanthanum based on total amount of catalyst. The lanthanum containing catalyst may be produced using any suitable method. A preferred method comprises impregnating a support with a lanthanum containing salt, and subsequently drying and calcining the impregnated support. After impregnation the impregnated support may be dried and subsequently calcined. Calcination is generally carried out at a calcination temperature from between 120° C. to 700° C. The catalyst activity can be increased even further if the catalyst is calcined at a temperature in the range of from 350° C. to 600° C. Preferred catalysts are described in PCT patent application No. PCT/EP02/12638.

A further catalyst which is especially suitable for use in step (b) of the present invention is a zinc supported catalyst. The support preferably is selected from the group consisting of Al₂O₃, SiO₂, MgO, TiO₂, ZrO₂, Cr₂O₃, carbon and mixtures thereof. The zinc supported catalyst may be prepared by impregnation of silica, alumina or mixtures of aluminium and magnesium (hydr)oxide with a zinc nitrate solution. Preferably, the zinc supported catalysts comprise at least 15% wt of zinc on a support having a surface area of at least 20 m²/g, more preferably at least 40 m²/g. Preferred catalysts are described in the patent applications claiming priority of European patent application No. 02256347.2.

A further catalyst which is preferably used is a catalyst consisting of alumina. Preferably, the alumina is gamma-alumina.

The hydrolysis of process step (b) is preferably carried out at a temperature of from 50° C. to 300° C., preferably of from 80° C. to 250° C., more specifically of from 100° C. to 200° C. The pressure can vary widely, and preferably is at most 100×10⁵ N/m², more specifically at most 60×10⁵ N/m², more specifically at most 40×10⁵ N/m². The pressure will usually be at least 1×10⁵ N/m², more specifically at least 5×10⁵ N/m².

Preferably, propylene glycol is separated from the second reaction mixture. The propylene glycol may be separated from the reaction mixture obtained in step (b) in any way known in the art. A preferred separation comprises distillation of the second reaction mixture, optionally followed by further distillation of one or more of the distillate fractions and/or bottom fractions. One or more of the fractions separated will have a high content of propylene glycol. Propylene glycol obtained by distillation will usually be sufficiently pure to use as such. If required, small amounts of by-products may be removed separately.

The present invention is further illustrated by the following example. The example is given for further illustration of the invention and is not limiting the invention.

### EXAMPLES

A 1 litre high-pressure autoclave reactor was loaded with 0.5 gram of MgO catalyst, to which were added propylene carbonate (PC), water, propylene oxide (PO) and 1,2-propanediol (monopropylene glycol, MPG). The amounts of the compounds (in mmole) are shown in Table 1. In Example 1, 0.5 gram of methyltributylphosphonium iodide catalyst (MTBPI) was added as well. The reactor was purged with N₂ or CO₂ and subsequently the autoclave was heated to 150° C. during the indicated number of hours. In some experiments, the reactor is additionally pressurized with CO₂.

At the end of the run, the autoclave was cooled to room temperature and the liquid was removed for off-line analysis by gas chromatography with sulfolane as the standard. The amounts of the compounds in the product (in mmole) are shown in Table 1. A known by-product is dipropylene glycol (DPG). A limited amount of further by-products was present in the product. The further by-products have not been analyzed. The results are shown in Table 1.

It is clear from Table 1 that propylene oxide present in the feed is converted while only a limited amount of dipropylene glycol is formed. Further reduction of the formation of dipropylene glycol is observed if the homogeneous catalyst for the conversion of propylene oxide into propylene carbonate is present during the hydrolysis of the propylene carbonate.

TABLE 1

| | | | Feed | | | | | Product | | | |
|---|---|---|---|---|---|---|---|---|---|---|---|
| | Time (h) | Catalyst | PC | MPG | H₂O | PO | CO₂ (bar) | PC | MPG | PO | DPG |
| 1 | 14 | MgO/MTBPI | 500 | 120 | 720 | 100 | 22 | 160 | 490 | 1 | 0 |
| 2 | 13 | MgO | 500 | 60 | 730 | 100 | 22 | 230 | 330 | 1 | 6 |
| 3 | 13 | MgO | 500 | 110 | 660 | 50 | — | 200 | 410 | 1 | 2 |
| 4 | 5 | MgO | 500 | 100 | 600 | 100 | — | 350 | 260 | 53 | 3 |
| 5 | 5 | MgO | 500 | 120 | 670 | 50 | — | 310 | 240 | 9 | 1 |

We claim:

1. A process for the preparation of propylene glycol from propylene oxide, which process comprises:
(a) contacting propylene oxide with carbon dioxide in the presence of catalyst and in the substantial absence of water to obtain a first reaction mixture comprising propylene carbonate; and,
(b) contacting at least part of the first reaction mixture with water in the presence of catalyst to obtain a second reaction mixture comprising propylene glycol and carbon dioxide,
in which process a substantial amount of propylene oxide is present in step (b).

2. The process of claim 1, in which the molar ratio of propylene oxide to propylene carbonate in step (b) is from 0.01:1 to 1:1.

3. The process of claim 1, in which the propylene oxide present in the feed of step (a) is substantially fully converted to propylene carbonate, and which process further comprises adding additional propylene oxide in step (b).

4. The process of claim 1, in which from 60% to 99% of the propylene oxide present in the feed of step (a) is converted in step (a) to propylene carbonate.

5. The process of claim 1, in which propylene glycol is separated from the second reaction mixture.

6. The process of claim 1, in which process step (a) is carried out with a homogeneous catalyst and step (b) is carried out with a heterogeneous catalyst.

7. The process of claim 6, in which process the homogeneous catalyst for process step (a) is also present in step (b).

8. The process of claim 6, in which the propylene oxide present in the feed of step (a) is substantially fully converted to propylene carbonate, and which process further comprises adding additional propylene oxide in step (b).

9. The process of claim 6, in which from 60% to 99% of the propylene oxide present in the feed of step (a) is converted in step (a) to propylene carbonate.

10. The process of claim 6, in which propylene glycol is separated from the second reaction mixture.
